(19) United States
(12) Patent Application Publication
(10) Pub. No.: US 2024/0382139 A1
(43) Pub. Date: Nov. 21, 2024

(54) A DEVICE FOR OBTAINING ERG SIGNALS WITH RELATED ARRANGEMENT AND METHOD

(71) Applicant: MACULASER OY, Helsinki (FI)

(72) Inventors: Ossi KAIKKONEN, Helsinki (FI); Teemu TURUNEN, Helsinki (FI); Jani TIRRONEN, Helsinki (FI); Jaakko NIEMINEN, Helsinki (FI)

(21) Appl. No.: 18/574,645

(22) PCT Filed: Jun. 27, 2022
(86) PCT No.: PCT/FI2022/050472

§ 371 Date: Dec. 27, 2023

(30) Foreign Application Priority Data
Jun. 28, 2021 (FI) 20215763

(51) Int. Cl. A61B 5/398

(52) U.S. Cl. CPC A61B 5/398

(57) ABSTRACT

A device for obtaining retinal ERG signals from a target area of the retina, the device comprising means for obtaining an electrical response signal from the target area, the device additionally comprising at least one light source configured to provide a stimulus beam towards the target area, wherein the stimulus beam is modulated to provide a plurality of single pulses of light, where consecutive single pulses are separated by predetermined intervals of which at least a portion differ from each other and/or wherein at least a portion of the single pulses of light comprise predetermined durations of which at least a portion differ from each other.

### TECHNICAL FIELD OF THE INVENTION

The invention relates to electroretinography (ERG) in general. More specifically, the invention relates to determining an ERG response from obtained ERG signals, where a stimulus beam provides a plurality of single pulses of light, wherein consecutive single pulses are separated by predetermined intervals, of which at least a portion differ from each other and/or wherein at least a portion of the single pulses of light comprise predetermined durations of which at least a portion differ from each other.

### BACKGROUND OF THE INVENTION

Electroretinography (ERG) is a method where electrical signals of the retina are recorded during exposure of the retina to stimulus, such as light flashes, which may be useful in various cases, such as in diagnosis of retinal diseases. When light falls on the retina, photoreceptors transduce the stimulus into an electrical signal through a biochemical cascade. The electrical signal is then passed onto other retinal neurons and finally to the brain through the optic nerve. This signaling produces a varying field potential, which can be recorded with electrodes placed on and/or near the eye and recorded as an ERG signal.

When a small portion of the retina is stimulated, the ERG signal is generated by the stimulated portion of the retina. This signal, termed the focal ERG (fERG) signal has an amplitude that depends on the extent of the stimulated area (the region or area of the retina that is stimulated, referred to herein also as a target area). Hence if the stimulated area is small, the signal-to-noise ratio (SNR) of the signal is low, and the stimulus must be repeated numerous times to enhance the SNR. fERG recording typically involves stimulating the target area of the retina repeatedly with light at a fixed rate (e.g. flashes of light that are repeated at a certain fixed frequency), and either investigating the signal in the frequency domain, or averaging the different repetition blocks of the signal to mitigate the effect of noise in the signal waveform.

When the retina is stimulated with the e.g. light flashes at a high frequency, the signal-to-noise ratio is enhanced, but as subsequent signals start to overlap, the waveform of the fERG signal does not represent the response to a single stimulus anymore. There is therefore a tradeoff between temporal resolution and obtaining an undistorted ERG signal without overlap effects that could truly be representative of the ERG response to each single stimulus, i.e. flash of light, for instance.

ERG may be used on its own, while it has also been discovered that ERG signals obtained during retinal heating (e.g. photothermal retinal therapies) could be used to determine the temperature of the retina, due to the temperature-dependence of the electrical signalling of the retina. An obtained ERG signal during retinal heating can therefore be indicative of a temperature (or at least a difference in temperature caused by retinal heating) that is occurring at the retina. The temperature determination related to heating could then be used e.g. for controlling the retinal heating so that the retinal temperature is kept at a desired level. However, the determination of temperature-related parameters based on ERG signals has been difficult and prior art methods have shown inaccurate determination of e.g. temperature or temperature difference exhibited by the retinal tissue based on recorded ERG signals during retinal heating.

### SUMMARY OF THE INVENTION

The object of the invention is to alleviate at least some of the problems in the prior art. In accordance with one aspect of the present invention, a device is provided for obtaining retinal ERG signals from a target area of the retina. The device comprises means for obtaining an electrical response signal from the target area, the device additionally comprising at least one light source configured to provide a stimulus beam towards the target area. The stimulus beam is modulated to provide a plurality of single pulses of light, where consecutive single pulses are separated by predetermined intervals of which at least a portion differ from each other and/or wherein at least a portion of the single pulses of light comprise predetermined durations of which at least a portion differ from each other.

In one other aspect of the invention, an arrangement for providing retinal ERG stimulus and heating is provided in independent claim 14. Here, the arrangement may be adapted to obtain an ERG signal (corresponding to the electrical response signal of the tissue), the ERG signal comprising overlapping ERG/electrical responses, wherein the arrangement may additionally be configured to determine a denoised ERG response from the ERG signal.

A method for obtaining an ERG signal is also provided according to independent claim 16.

Utilizing the present invention, an ERG response may be determined where the ERG response may more efficiently represent a true response of the retinal tissue at the target area to each single pulse of light due to overlap of consecutive responses in the recorded ERG signal being avoided or reduced, which is enabled by the provided uneven intervals between stimuli and/or uneven durations of the stimuli. This may be beneficial in any further processing of the ERG response data, such as determination of other parameters based thereon.

In this text, the term “ERG signal” generally refers to an electrical signal that is recorded during the retinal stimulation, which is representative of a response of the retinal tissue to the stimulation light. The term “ERG response” refers either to an actual/true response of the tissue to the stimulation or to a determined or extracted response to a single stimulus, e.g. an impulse response that is determined from the ERG signal. A “true ERG response” or “electrical response” thus generally refers herein to the actual response of the tissue to the single pulses and a plurality of the consecutive electrical responses constitute the ERG signal. The “determined ERG response” then refers to the e.g. impulse response extracted from the ERG signal using e.g. computational means.

The stimuli (e.g. light pulses) may yet be delivered with intervals selected such that a beneficial signal-to-noise ratio of a determined ERG response may be provided, e.g. a SNR may be increased or maximized. Yet, the present invention provides a device and method that may be used in connection with ERG methods such as focal ERG where SNR is relatively low due to, e.g., stimulation of a single small target area, compared to other methods, for instance full-field ERG with larger target area stimulated.

The intervals and/or durations may be selected to provide an ERG signal exhibiting selected overlap of electrical responses that are related to consecutive single pulses. Note that the selected overlap is overlap of electrical responses, whereas the single pulses of the stimulus light may be non-overlapping. The intervals and/or durations may be selected such that the consecutive single pulses are substantially non-overlapping and such that an ERG signal exhibiting selected overlap of electrical responses related to said consecutive single pulses is provided. Preferably, the consecutive single pulses are provided by one light source. Alternatively, more than one light source can be used. The selected overlap may comprise overlap of consecutive electrical responses in essentially different phases.

In one embodiment, the selected overlap may provide a selected waveform for a determined ERG response obtained from an ERG signal comprising containing a plurality of overlapped electrical ERG responses optionally through an averaging method, such as cross-correlation between the recorded ERG signal and a stimulus series used to modulate the stimulus beam. An ERG impulse response may also be determined through signal averaging. The selected waveform may essentially correspond to the electrical responses or differ from them by under a selected amount. The ERG response determination may also comprise removing or reducing artifacts from the obtained ERG signal based on one or more artifact-rejection criteria.

In one embodiment, ERG may be used in combination with therapeutic retinal heating and the determined ERG response may be used to determine one or more temperature-related parameters, such as parameters indicative of a temperature of the retinal tissue.

As a more accurate representation of the true ERG response waveform may be determined through the present invention, the determined temperature-dependent parameter(s) may thus also be more accurate.

A device combining laser retinal heating and ERG stimulation is known from the prior art, where a high frequency square wave (e.g. 41 Hz) was used in modulation of a stimulus beam to acquire ERG signals to be used in determination of ERG responses and subsequent retinal temperature estimation. In this approach, the retinal temperature information cannot be extracted from the signal with high accuracy.

It has been found that if the frequency of the square wave is reduced sufficiently, temperature information could be more accurately retrieved from the ERG signal but the signal-to-noise ratio of the determined ERG response would suffer due to a lower number of electrical responses elicited in the retina per unit time.

The inventors have found that in the prior art cases where high frequency is used for modulation of stimulus beams at even intervals, the inaccuracy in subsequently determined temperature-related parameters is due to the overlap of consecutive electrical responses in the ERG signal. When ERG is used for retinal temperature determination, often the a- and b-waves of the ERG signal are relevant for analysis. If a high stimulation frequency and constant intervals between stimuli such as light flashes is used, the overlap of late ERG waveforms on the a- and b-waves of the subsequent response may be coupled into an ERG response determined from a series of ERG responses through a technique such as signal averaging, causing error in determining retinal temperature from the signal. The inventors then discovered that utilizing a stimulation protocol where the intervals between stimulus pulses and/or the durations of the stimulus pulses are varied, the information related to temperature changes in the retina may be determined more accurately and with superior signal-to-noise ratio compared to utilizing even stimulus pulse intervals and stimulus pulse durations. It was then also considered that this inventive stimulation protocol could also be beneficial in other use cases where ERG responses determined from ERG signals are utilized, at least in those where similar methods are used for extraction of the determined ERG signals.

A heating beam may in some embodiments of the invention be directed to at least the target area (the same area to which the stimulus beam is directed) and at least two measured signals related to an ERG signal of the retina may be obtained at two different time intervals, optionally with different heating powers, from which two ERG responses may be determined. At least one parameter that is indicative of a change in temperature of the retinal tissue may be determined based on the determined ERG responses.

From an indication of a determined change in temperature, a temperature increase of the retina per unit of heating power may be determined.

In some embodiments, a heating power used for retinal heating may be controlled based on determined temperature-related parameters, such as a determined temperature increase of the retina per unit of heating power.

With the present invention, a retinal temperature or change in retinal temperature may be determined more accurately than in the prior art. A heating power for heating the retina may thus also be more efficiently controlled, possibly enabling safer and/or more effective retinal heating devices and methods. For example, a personalized heating power may be delivered more effectively when the temperature increase per unit heating power may be more accurately determined for an individual.

The intervals and/or durations may be selected to provide an ERG signal from which the ERG response may be determined such that waveform distortion arising from electrical responses of the tissue to at least two consecutive single pulses being overlapped in said ERG signal is reduced or maintained under a threshold amount and/or coupling of the noise in the recorded ERG signal to the extracted ERG response is reduced or maintained under a threshold amount. If a coupling of the noise in ERG signal to the extracted ERG response is additionally reduced in embodiments of the invention, the determined ERG response may be more accurately representative of the true response of the tissue to the stimulus compared to cases where coupling of the ERG signal to noise is not reduced.

In some embodiments, a reference ERG response may be obtained, and the intervals and/or durations may be selected to provide an ERG signal from which the ERG response may be determined such that the ERG response differs from the reference ERG response by under a threshold amount.

In one other embodiment, the intervals and/or durations of stimulus pulses may be selected to provide an ERG signal exhibiting selected overlap of electrical responses (the true/elicited ERG responses) that are related to consecutive single pulses. The selected overlap may comprise overlap of consecutive true ERG responses in essentially different phases.

The intervals and/or durations may be between 10 and 200 milliseconds, advantageously between 20 and 70 milliseconds. As the stimulus intervals (and/or durations) are shortened, the amplitude of the ERG signal may begin to drop. Consequently, stimulating the retina at excessively short intervals may result in diminishing ERG signal amplitudes and a lower signal-to-noise ratio. Therefore, a minimum interval or duration may be selected as a lower limit for a range of a duration of an interval between consecutive pulses of light or a duration of a single pulse of light. It has been observed that a minimum interval or duration of above 10 ms, advantageously above 20 ms, may provide preferred SNR.

However, when the stimulus intervals (durations between separate pulses or durations of single pulses) are increased beyond a certain duration, the amplitude of the ERG signal no longer increases. Using longer stimulus intervals or durations results in fewer stimuli being presented per unit time (decreased stimulus rate/frequency), resulting in a lower signal-to-noise ratio. Accordingly, a maximum interval or duration may be selected as an upper limit for a range of a duration of an interval between consecutive pulses of light or a duration of a single pulse of light. It has been observed that a maximum interval or duration of below 200 ms, advantageously below 70 ms, may provide optimal or maximized SNR.

The intervals and/or durations may be selected such that minimized or reduced signal contribution is provided at or near a mains frequency, such as 50 or 60 Hz, and/or its harmonic frequencies in the frequency band where the ERG signal has spectral content (e.g. 5-300 Hz), i.e. the periodogram of the stimulus output may have clear minima around the aforementioned frequencies. When the stimulus series does not have content at a certain frequency, noise at that frequency may be attenuated in the ERG response to a single stimulus. When the stimulus series (provided sequence of single pulses of light) does not have content at a certain frequency band, the resulting determined ERG response also does not have content at the same frequency band, and applying a notch filter of this frequency band on the signal may have a lesser effect on the ERG signal. When the intervals and/or durations of the single pulses have been selected such that minimized or reduced signal contribution is provided at or near a mains frequency, such as 50 or 60 Hz, and/or its harmonic frequencies in the frequency band where the ERG signal has spectral content, the ERG impulse response determination may additionally comprise removing from the analysis or weighting differently also ERG signal data that would otherwise be considered suitable for the analysis in order to minimize or sufficiently reduce the contribution of noise in mains-related frequencies.

The intervals and/or the ordering of the varying intervals between pulses and/or durations of pulses may be selected to provide a stimulation pulse train with broad spectral content. An impulse ERG response may be determined with reduced or minimized waveform distortion (reduced deviation from an actual response of the retina) by stimulating the retina with stimulus light modulated by white noise (ideally broad spectral content), and extracting the ERG response by computing the cross correlation function between the white noise used to modulate the stimulation and the recorded ERG signal. However, white noise stimulation is not ideal for ERG recording with low signal amplitude relative to noise, such as local ERG recordings from a small retinal target area, as it results in a lowered signal-to-noise ratio, due to a low amplitude of ERG signal obtained. However, the intervals between light pulses and/or the duration of light pulses may be selected to produce a stimulus series with broad spectral content, which may result in a waveform of an extracted/determined ERG response to a single stimulus (e.g. an impulse response) that is distorted minimally or to a lesser degree than with e.g. using stimuli that are evenly spaced while maintaining the superior SNR of pulsed stimulation compared to white noise stimulation.

In some embodiments, the number of intervals and/or durations temporally differing from each other may be for instance above 4.

The longest interval and/or duration may be 10 to 150%, advantageously 40 to 100%, longer than the shortest interval and/or duration. It may be advantageous to avoid excessively long stimulus intervals or stimulus durations, as the time required for recording of the ERG signals is increased. However, when the difference in stimulus intervals or stimulus durations is too short, the waveform distortion in determined ERG response caused by signal overlap may become larger than desired. The aforementioned differences between longest and shortest intervals or durations have been observed as giving preferred results.

In some further embodiments of the invention, the intervals and/or durations may be approximately evenly spaced between the shortest and the longest interval and/or duration. It has been observed that this may provide an advantageous stimulus series exhibiting characteristics that may lead to e.g. determination of an ERG response that has reduced waveform distortion.

In some embodiments, sets of pulses may be provided such that a first set of single pulses of light is provided, preferably where each interval and/or duration differs from each other in length, and an ordering of the single pulses and respective intervals and/or durations is varied at least once to provide at least a second set of single pulses. The at least second set of single pulses may be provided following the first set of single pulses to provide at least one extended set of single pulses of light.

The single pulses of light may be impulse-like pulses of light or non-impulse-like pulses of light. In the case of non-impulse-like pulses of light, the single pulses may be separated by predetermined intervals, the duration of which at least a portion differ from each other and/or the durations of at least a portion of the single pulses of light may differ from each other by predetermined amounts. The term “impulse-like” may refer to a pulse of light that has a duration which is under a certain threshold such that under this threshold, the duration of the light pulse essentially does not affect the form of the light pulse signal, if the same amount of photons is being produced. An impulse-like pulse of light could e.g. have a duration of 1 ms.

Selection of a stimulus series may herein refer to selection of characteristics of the provided set of single light pulses, such as selection of the intervals and/or durations and/or an order of the single pulses and associated intervals and/or durations.

The exemplary embodiments presented in this text are not to be interpreted to pose limitations to the applicability of the appended claims. The verb “to comprise” is used in this text as an open limitation that does not exclude the existence of unrecited features. The features recited in depending claims are mutually freely combinable unless otherwise explicitly stated.

The novel features which are considered as characteristic of the invention are set forth in particular in the appended claims. The invention itself, however, both as to its construction and its method of operation, together with additional objects and advantages thereof, will be best understood from the following description of specific example embodiments when read in connection with the accompanying drawings.

The presented considerations concerning the various embodiments of the device and arrangement may be flexibly applied to the embodiments of the method mutatis mutandis, and vice versa, as being appreciated by a skilled person.

### BRIEF DESCRIPTION OF THE DRAWINGS

Next the invention will be described in greater detail with reference to exemplary embodiments in accordance with the accompanying drawings, in which:

FIG. 1 schematically shows at least a portion of an exemplary device and arrangement according to one embodiment of the invention,

FIG. 2 schematically illustrates at least a portion of one more exemplary device and arrangement according to an embodiment of the invention,

FIG. 3 depicts a theoretical example of how different stimulus series may result in differing ERG signals and how the determined ERG response may be affected,

FIG. 4 shows a periodogram of an exemplary stimulus series according to an embodiment of the invention,

FIG. 5 shows ERG responses obtained with a prior art stimulus pulse series,

FIG. 6 shows ERG responses obtained with a stimulus pulse series according to an embodiment of the invention,

FIG. 7 depicts determined ERG responses during retinal heating using a prior art stimulus pulse series and temperature elevations determined from the determined ERG responses,

FIG. 8 shows determined ERG responses during retinal heating using a stimulus pulse series according to the present invention and temperature elevations determined from the determined ERG responses, and

FIG. 9 illustrates a flow chart of a method according to one embodiment of the invention.

### DETAILED DESCRIPTION

FIG. 1 shows at least a portion of an exemplary device 100 or arrangement 200 according to one embodiment of the invention. The device 100 comprises at least one light source 102 that is configured to provide a stimulus beam, where the stimulus beam is adapted to be directed towards a target area of a retina of an eye 104. The light source 102 is configured to provide the stimulus beam such that the stimulus beam or stimulus light is modulated to provide a plurality of single pulses of light, where consecutive single pulses are separated by predetermined intervals of which at least a portion differ from each other and/or wherein at least a portion of the single pulses of light comprise predetermined durations of which at least a portion differ from each other.

Predetermined intervals may refer to intervals that are determined prior to delivery of the stimulation sequence or ones determined/generated for instance during the stimulation sequence, e.g., with the help of a random number generator.

The light source 102 may for instance be a light emitting diode (LED) light source or laser light source. The light source 102 is preferably configured to provide an essentially focused beam of electromagnetic radiation to the target area of the retina. The device 100 may be configured to stimulate the target area of the retina with the stimulus beam to elicit a focal ERG signal.

The target area may be a single area, covering a surface area that is relatively small, e.g., smaller than the target area usually used in full-field ERG. The target area may comprise a circular area on the retina with a diameter of 1-12 mm, preferably 1-10 mm, and even more preferably 1-7 mm. The target area could have any other shape, such as, for example, a ring- or annular-shape, an oval, or a rectangle. The target area may comprise one or more surface areas of the retina, where in case of more than one area, such areas may be disconnected, simply connected or non-simply connected. The target area can be the same to all single pulses, i.e., the one or more areas comprised within the target area can be the same for all single pulses. Single pulses of a pulse series may also have different target areas.

The stimulus beam light source 102 may be a light-emitting diode (LED) light source configured to provide a stimulus beam having wavelength of 500-600 nm, e.g. about 555 nm. Red and green cone cells have similar sensitivity at a wavelength of 555 nm, therefore a stimulus beam exhibiting a wavelength close to this may stimulate both cell types similarly. The stimulus beam may comprise white light, which may stimulate all retinal cone cells equally. “Stimulus beam” may be used herein to refer to one or more of the single pulses of light that are provided by the light source 102, which may be impulse-like pulses of light or non-impulse-like pulses of light. The consecutive single pulses of light are preferably substantially not overlapping. Advantageously, the consecutive single pulses are provided by one light source.

The intervals and/or durations may be selected to provide an ERG signal from which the ERG response may be determined such that waveform distortion arising from ERG responses to at least two consecutive single pulses being overlapped in said ERG signal is reduced or maintained under a threshold amount and/or coupling of the ERG signal to noise is reduced or maintained under a threshold amount.

The intervals and/or durations may for instance be between 10 and 200 milliseconds, advantageously between 20 and 70 milliseconds.

The intervals and/or durations may additionally or alternatively be selected such that minimized or reduced signal contribution is provided at or near a mains frequency, such as 50 or 60 Hz, and its harmonic frequencies in the frequency band where the ERG signal has spectral content.

The intervals and/or durations and/or an order of the single pulses and associated intervals and/or durations may be selected to provide a stimulation pulse train with broad spectral content.

The longest interval and/or duration may be 10 to 150%, advantageously 40 to 100%, longer than the shortest interval and/or duration.

The intervals and/or durations may be approximately evenly spaced between the shortest and the longest interval and/or duration.

Sets of pulses may be provided such that a first set of single pulses of light is provided, preferably where each interval and/or duration differs from each other in length, and an ordering of the single pulses and respective intervals and/or durations is varied at least once to provide at least a second set of single pulses. The at least second set of single pulses may be provided following the first set of single pulses to provide at least one extended set of single pulses of light.

The device 100 additionally comprises means 106 for obtaining an ERG signal from the target area of the retina. The means 106 for obtaining an ERG signal may comprise (not depicted) an ERG electrode placed on the surface of the eye 104 of the target subject and a reference electrode placed somewhere else in contact with the body of the target subject, e.g. on the skin near the eye. Changes in potentials (voltage) between the electrodes may then be registered as an ERG signal.

The device 100 may also comprise or be used in connection with one or more optical elements 108, which may comprise one or more lenses, mirrors, prisms, and/or filters for focusing or otherwise handling the stimulus beam and/or any other electromagnetic beams that are to be directed towards the eye 104.

The device 100 may be comprised in an arrangement 200, which may additionally comprise at least one processing unit 110 such as a microprocessor, microcontroller and/or a digital signal processor or the device 100 may be considered to comprise also a processing unit 110. The processing unit 110 may be configured to execute instructions embodied in a form of computer software stored in a memory, which may refer to one or more memory chips, for example, separate or integral with the processing unit 110 and/or other elements. The memory may store various further data in addition to mere program instructions. It may, for example, host a number of data repositories such as databases accommodating information such as information regarding previously measured ERG signals (e.g. reference ERG responses).

The software may define one or more applications for executing the activities described herein. A computer program product comprising the appropriate software code means may be provided. It may be embodied in a non-transitory carrier medium such as a memory card, an optical disc or a USB (Universal Serial Bus) stick, for example. The software could also be transferred as a signal or combination of signals wired or wirelessly from a transmitting element to a receiving element.

The processing unit 110 may be configured to determine an ERG response based at least on the obtained ERG signal. The determined ERG response may be an ERG impulse response. The determined ERG response may be extracted from the obtained ERG signal, such that the determined ERG response is more accurately representative of the response of the retinal tissue to the provided stimulus beam than the recorded ERG signal. The ERG response may be determined such that a more accurate response of the retinal tissue (better representative of a true ERG response, especially with regards to the a- and b-waves) to a single pulse of stimulating light may be extracted from the ERG signal.

The extraction of the determined ERG response from the obtained ERG signal may include removing or applying a lower weight to artifacts from the ERG signal based on artifact-rejection criteria, where the artifacts relate to strong, short-lasting muscular activity or burst(s) of external electromagnetic artifacts. In other words, in some cases, it may be beneficial to exclude ERG signal data corresponding to some single pulses of light from the analysis, or, e.g., to have a lower weight for those data points in the analysis if responses are combined by calculating their weighted average. The ERG signal data corresponding to a single pulse of light may be defined, e.g., as the data within a certain time interval after the single pulse of light (e.g., within 0-40 ms after a single pulse or, e.g., within 0-100 ms after a single pulse). According to another definition, also data preceding a single pulse may correspond to that single pulse. A situation when this kind of handling of data may be beneficial may occur, e.g., if the ERG signal is contaminated by artifacts, e.g., strong, short-lasting muscular activity (electromyography, EMG) or burst(s) of external electromagnetic artifacts; in this case, the corresponding segments are ideally removed from the averaged ERG signal, as, otherwise, such artifacts may distort the averaged ERG signal (typically, artifacts to be of concern may be of high amplitude, exceeding a threshold value, such as, e.g., at least 30% above the value of neighboring or standard values) and thus averaged responses may have features of artifactual origin, if artifacts are not accounted for. Similarly, if it is known, e.g., that ERG signal data corresponding to certain single pulses of light is of different origin than the rest of the data (e.g., if those single pulses of light were delivered to a different area in the eye, e.g., because of movement of the eye or a fundus lens), it may be beneficial to exclude the data corresponding to those single pulses of light from the analysis.

To identify segments of data and the corresponding single pulses of light that could be removed from the analysis (or, e.g., weighted differently) one may utilize various techniques. One may, e.g., estimate/monitor where single pulses of light are delivered, e.g., through a camera system or by monitoring the placement of optical elements (including the eye) on the path of the single pulses of light. To identify artifactual data segments one may, e.g., high-pass filter the signal (e.g., with a high-pass filter having a cut-off frequency in the range 1-20 Hz), rectify the signal, optionally raise the rectified signal to a suitable power (e.g., to the power of 0.5, 1, 1.5, 2, or 3), and calculate a moving average (or, e.g., a moving median) of the processed signal over a time window of suitable length (the window length may be, e.g., 50 ms or selected from, e.g., the range of 5-500 ms). One may then mark data artifactual, e.g., at or near time instants where the amplitude of the processed moving-average signal exceeds a threshold value. This threshold value may be, e.g., a fixed value or determined, e.g., based on the statistics of the analyzed signal or the statistics of suitable reference data.

The stimulation protocols or stimulus series according to embodiments of the invention enable the acquisition of ERG signals with a higher rate, improving the SNR with a given temporal resolution, with the acquired ERG signal exhibiting signal overlap due to overlap of electrical signals that are related to response of tissue to separate, consecutive stimulating light pulses such that the signal overlap may be mitigated in an ERG response determined from the ERG signal. The determined ERG response may be less distorted than in the case of even stimuli, and more accurately represent an actual or true ERG response.

When the stimulus beam is modulated to provide a plurality of single pulses of light, where consecutive single pulses are separated by predetermined intervals of which at least a portion differ from each other and/or wherein at least a portion of the single pulses of light comprise predetermined durations of which at least a portion differ from each other, the ERG response may be determined utilizing an appropriate method to obtain a determined ERG response that is of higher quality or more accurately represents an actual ERG response of the tissue than if the stimulus beam would provide single pulses of stimulating light at even intervals.

An appropriate selection of the characteristics of the stimulus series may be important to be able to obtain a determined ERG response to a single stimulus from the ERG signal with a plurality of overlapped responses. Additionally, the selection of appropriate stimulus intervals may affect how different frequencies of the ERG signal and signal noise are coupled into the extracted/determined ERG response to a single stimulus.

The determined ERG response may be obtained for instance through signal averaging or cross-correlation between the ERG signal and the stimulus series. When ERG signals are elicited with stimulus impulses with uneven intervals, the impulse response can be extracted through computational methods, such as signal averaging or cross correlation between the recorded response series and the stimulus series. Let x(n) be the recorded ERG signal and let s(n) be the stimulation series, where the index n runs from 1 to N. s(n)=1 means that the device delivers a stimulus pulse at time t(n), and s(n)=0 means that the device does not deliver a stimulus pulse at t(n). The ERG response to a single stimulus y(n) may be extracted through cross correlation, which is shown in the following equation:

$$y ( n ) = \frac{1}{{\sum }_{m = 1}^{N} s ( m )} {\sum }_{i = 1}^{N - n} s ( i ) x ( i - n ) \qquad ( 1 )$$

When cross correlation is used to extract an ERG response from an ERG signal comprising a plurality of electrical responses of the tissue, the overlap of electrical responses (true ERG responses) creates a waveform distortion corresponding to the averaged waveform of the overlapped electrical response tails of the previous electrical responses. When a constant stimulation frequency is used, the electrical response tails are always in the same phase relative to the subsequent response, and the resulting determined ERG response is the sum of an electrical response and the overlapped tail of the previous electrical responses. When the intervals between stimuli are varied, the response tails of the previous response overlapping with a subsequent response are in different phases, resulting in an attenuated average waveform.

In order to attenuate a broad range of response tail waveforms, it may be beneficial for a stimulation sequence (or stimulation series) to contain a broad range of stimulus intervals instead of, e.g. only repeating a small number of intervals, or a broader set of intervals, which are strongly clustered.

The processing unit 110 may in some embodiments be used to control the stimulus light source 102 to provide the selected stimulus series or the device 100 may comprise a separate controller unit for controlling the stimulus light source 102.

In some embodiments of the invention, an arrangement 200 may be provided, where the arrangement comprises a device 100 and controller unit 112 that is configured to control the stimulus light source 102. The processing unit 110 and the controller unit 112 may be provided as separate entities that may be in communication with each other, but they may also be provided as one processing entity that is configured to perform the functionalities as described herein.

A controller unit 112 may be configured to select a suitable stimulus series for a specific use case, e.g. based on a reference ERG response as described below.

In one embodiment, a device 100 may however be configured to provide a selected stimulus series via the stimulus light source 102, where the stimulus series may be obtained e.g. via a database through a processing unit or controller unit, or predetermined, previously selected stimulus series may be stored for instance in a memory unit associated with a device 100.

In one embodiment of the invention, the processing unit 110 and/or controller unit 112 may be configured to obtain one or more reference ERG responses. The reference ERG responses may correspond to ideal ERG responses or may be previously measured ERG responses. The reference ERG responses may provide information on an actual ERG response of the retinal tissue to a known stimulus series, such that the reference ERG responses are known to be essentially undistorted due to overlap between responses to consecutive signals, i.e. the reference ERG responses may be representative of responses to single stimuli. A reference ERG response may for instance be obtained using a stimulus series providing single pulses of light at low frequency/repetition rate.

The reference ERG response may represent an ERG response of a target population. The stimulus series may be selected based on the use case to provide a suitable series for a target subject, the retina of which is to be stimulated, so that a determined ERG response may more accurately reflect the response of the retina to the stimulation, where the target subject belongs to the target population.

The selection of the stimulus series may be based on optimization or selection of different characteristics of the stimulus series (e.g. interval and/or duration as described hereinbefore, order of the intervals and/or durations) to yield an ERG signal that may be used to determine an ERG response from which the ERG response to a single pulse may be more accurately determined.

The ERG signal that may be obtained in a measurement scenario may be estimated by the processing unit 110/112 to be able to select a suitable stimulation series.

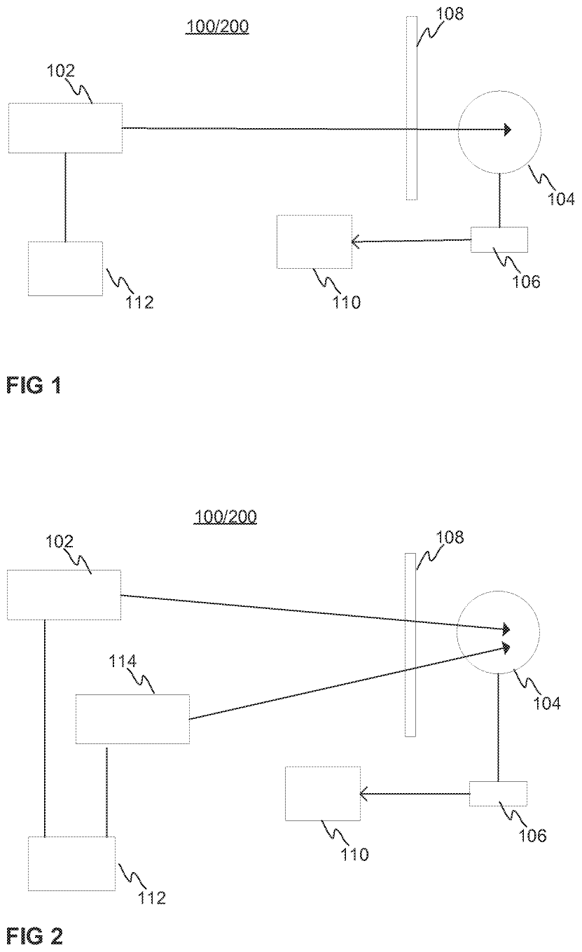

FIG. 2 shows one further embodiment of a device 100 and arrangement 200. In this embodiment, the device 100 and arrangement 200 may otherwise correspond to that of FIG. 1, but in addition the device 100 comprises at least a heating system for elevating the temperature of at least the target area of the retina. A heating system may for instance comprise a heating light source 114, such as laser, configured to provide a heating beam that is directed towards the target area of the retina. The heating beam may comprise wavelength in the near-IR area. Wavelengths of light comprised in the heating beam may be 700-1000 nm, while the heating beam may be provided by a heating light source 114 that is a fiber coupled diode laser. A heating system may also be implemented through other means, e.g. a heating system may utilize heating via ultrasound.

In embodiments where retinal heating is conducted, the stimulus beam may be of equal size or smaller than a heating beam used for heating at least the target area. The stimulus beam may e.g. have a beam/spot diameter that is about 75% of a treatment beam diameter.

In the embodiment of FIG. 2, the processing unit 110 may be configured to, after determining the ERG response from an obtained ERG signal, determine at least one temperature-related parameter based on the determined ERG response.

For instance, the heating light source 114 may be configured to provide at least two different heating powers for heating the target area (of which a first heating power could essentially correspond to zero heating power). The stimulus light source may be configured to provide at least one selected stimulus series for stimulating the target area at the at least two heating powers.

A device 100 or arrangement 200, specifically at least one processing unit 110, may be configured to obtain or receive ERG signals at the two different heating powers and determine ERG responses corresponding to the at least two different heating powers, e.g. through cross-correlation.

The determined ERG responses may be used to determine at least one temperature-related parameter corresponding to the at least two heating powers, where the temperature-related parameter is indicative of a temperature of the target area of the retina. The determined temperature-related parameters may be utilized to determine a change in temperature of the target area of the retina that occurs between the two different heating powers.

In one exemplary method for determining the temperature difference exhibited by the retinal tissue relating to two different heating powers based on respective determined ERG (impulse) responses, changes in kinetics, such as time delays of the determined ERG impulse response, may be analyzed. The ERG impulse response typically has one or more peaks, and the time delay between the impulse stimulus and a peak in the ERG response waveform is an example of a signal feature that can be used in temperature difference determination. The amount of time shift in the peak(s) between ERG impulse responses determined e.g. with and without (laser) heating is/are proportional to the extent of temperature elevation in the retina, and can be used to determine changes in retinal temperature.

Another exemplary method for determining the temperature difference from two ERG impulse responses is carried out by compressing/expanding the time axis of ERG signal of one or both of them, with the compression origin set to the time of the flash stimulus, and determining the amount of time-axis compression/expansion that maximizes the correlation between the responses. The amount of time axis compression/expansion producing the maximum correlation between the responses can be used to determine the amount of temperature elevation caused by the retinal heating and can be used to determine changes in retinal temperature of the target area.

With the present invention and the provided selected stimulus series, the determined temperature-related parameters or e.g. determined temperature changes may be determined more reliably and/or accurately than in the prior art due to the possibly more accurate determined ERG responses.

In some embodiments, a controller unit 112 may be configured to control, in addition or alternatively to the stimulus light source 102, the heating light source 114 (or other heating system). The controlling may be based on the determined temperature-related parameters.

In one embodiment, the processing unit 110 may be configured to determine a temperature elevation of the retinal tissue per unit of heating power. This may be used to control the heating light source 114 to provide a selected heating power that will result in a desired temperature of the retinal tissue at the target area or a desired temperature increase of the retinal tissue at the target area.

A processing unit 110 and/or control unit 112 may optionally also be configured to perform a calibration procedure based on the determined temperature-related parameter(s), so that a personalized heating power may be delivered to the target area that results in a desired temperature elevation of the retina for a specific target subject. With the invention, personalized heating powers or any other e.g. values or parameters that are determined based on the determined temperature-related parameters or determined ERG responses may be more reliable and thus also more safe and/or efficient for the target subject.

The devices 100 may be used in connection with a fundus imaging system (which may in some embodiments also be realized as part of a device). The fundus imaging system IS may for instance be a fundus camera or a scanning laser ophthalmoscope.

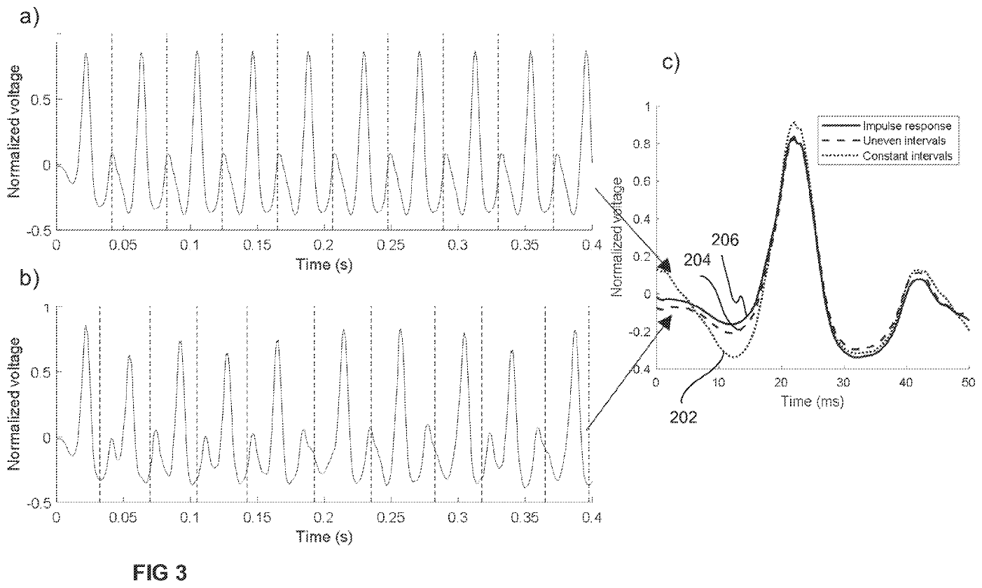

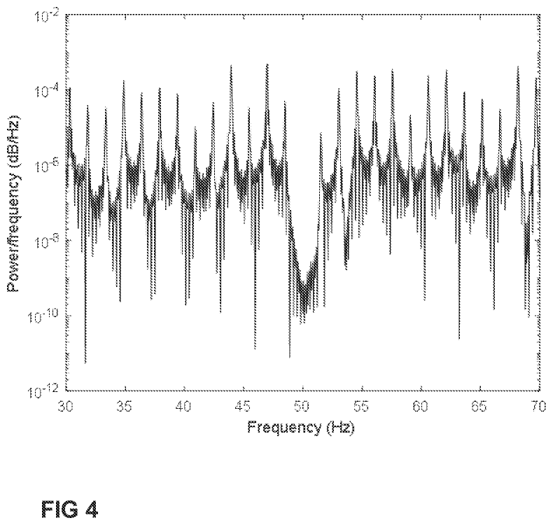

FIG. 3 shows a theoretical example of how different stimulus series may result in differing ERG signals and how the determined ERG response may be affected. FIG. 3 depicts at a) an example of an ERG signal that may be obtained with a stimulus series known in the prior art, where stimulating light pulses are delivered at the target area of the retina where pulses of light are separated by even intervals (constant stimulation frequency with the same average repetition frequency). FIG. 3 shows at b) an example of an ERG signal that may be obtained with a stimulus series according to the present invention, where e.g. pulses of light are provided with uneven intervals between the pulses. The time points of stimuli are marked with vertical dashed lines.

FIG. 3 further shows at c) determined ERG responses corresponding to an impulse response to a single light pulse that are determined from the ERG signals of a), shown at 202 with dotted line for the even/constant stimulus intervals, and b), shown at 204 with dashed line for the uneven stimulus intervals according to the invention.

At 206 with solid line, a reference ERG response is shown, where the reference ERG response is one that is known to be essentially free from waveform distortion due to overlapping consecutive electrical responses in an ERG signal, and thus represents a true ERG response.

From c) at FIG. 3, it may be seen that the ERG response 202 determined from the ERG signal of a) also exhibits a clear waveform distortion or difference in waveform compared to the true impulse ERG response, while the ERG response 204 determined from the ERG signal of b) exhibits lesser waveform distortion and is closer to the true ERG response than the response 202 determined from the ERG signal of a).

The intervals and/or durations in a stimulus series may be selected to provide an ERG signal from which the ERG response may be determined such that waveform distortion arising from ERG responses to at least two consecutive single pulses being overlapped in said ERG signal is reduced or maintained under a threshold amount compared to an obtained reference ERG signal and/or coupling of the ERG signal to noise is reduced or maintained under a threshold amount.

Advantageous characteristics of the stimulus series may be selected based on the use case.

A stimulus series s(n) may be represented as a series of stimulus intervals Hₘ. The intervals may be converted into a stimulus series by adding ones to a vector of zeros in a way that the intervals between the ones match the intervals in Hₘ.

For example, a light pulse interval series H=(32.5, 37.5, 35, 37.5, 50, 42.5, 47.5, 35, 47.5, 32.5, 50, 40, 42.5, 45, 45, 40) ms has an average frequency of 24.24 Hz and may elicit an ERG signal that may be used to reconstruct/determine the ERG response.

In one embodiment, the intervals and/or durations may be selected such that minimized or reduced signal contribution is provided at or near a mains frequency, such as 50 or 60 Hz, and its harmonic frequencies in the frequency band where the ERG signal has spectral content. This is shown in FIG. 4, giving a periodogram of an exemplary stimulus series according to an embodiment of the invention. Here, the periodogram shows clear minima at 50 Hz, preventing 50 Hz power line frequency from being coupled into the extracted impulse response. In embodiments where the intervals and/or durations of the single pulses have been selected such that minimized or reduced signal contribution is provided at or near a specific frequency, such as at or near a mains frequency, such as 50 or 60 Hz, and/or its harmonic frequencies in the frequency band where the ERG signal has spectral content, the ERG impulse response determination may additionally comprise excluding from the analysis or, e.g., weighting differently, also ERG signal data that would otherwise be considered suitable for the analysis e.g., data within artifact-free segments or data corresponding to single pulses of light that have been delivered to the correct location. Such additional exclusions (or, e.g., adjusted weighting) may be advantageous in maintaining a sufficient level of reduction of the signal at mains-related frequencies.

For example, if the intervals of single pulses of light have been selected so that they minimize the contribution of noise in mains-related frequencies, it may turn out that if only single pulses corresponding to the applied exclusion criteria (e.g., artifactual data, wrong signal origin) were excluded, the remaining single pulses of light would not minimize the contribution of noise in mains-related frequencies optimally (i.e., the intervals and/or durations of the remaining pulses may not be optimal for minimizing the signal contribution at mains-related frequencies). In such a situation, it may thus be beneficial to exclude also additional single pulses of light from the analysis so that the remaining single pulses of light have intervals and/or durations that allow maintaining a desired level of minimization or reduction of signal at mains-related frequencies.

Similarly, if, instead of fully excluding bad data (e.g., artifactual data or wrong signal origin) from the analysis, one applies, e.g., a different weighting for such data, the weighting may compromise the minimization or reduction of signal contribution at mains-related frequencies if the weighting is applied only to data corresponding to a certain subset of the original single pulses. In such a situation, it may thus be beneficial to apply weighting also to data that would otherwise remain unaffected to achieve a desired level of reduction of signal contribution at mains-related frequencies.

The intervals and/or durations may be between 10 and 500 milliseconds, advantageously between 15 and 200 milliseconds, and more advantageously between 20 and 70 milliseconds.

Additionally or alternatively, the intervals and/or durations and/or an order of the single pulses and associated intervals and/or durations may be selected to provide a stimulation pulse train with broad spectral content.

Further, the longest interval and/or duration may optionally be 1 to 400%, advantageously 10 to 150%, more advantageously 40 to 100%, longer than the shortest interval and/or duration.

Yet, in some further embodiments of the invention, the intervals and/or durations may additionally or alternatively be approximately evenly spaced between the shortest and the longest interval and/or duration.

In some embodiments, sets of pulses may be provided such that a first set of single pulses of light is provided, preferably where each interval and/or duration differs from each other in length, and an ordering of the single pulses and respective intervals and/or durations is varied at least once to provide at least a second set of single pulses. The at least second set of single pulses may be provided following the first set of single pulses to provide at least one extended set of single pulses of light.

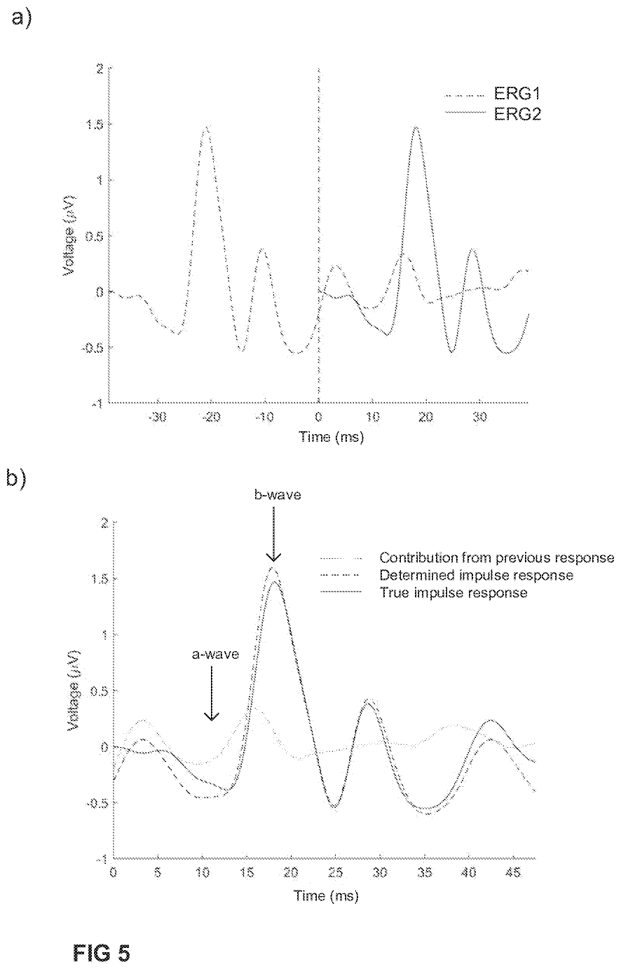

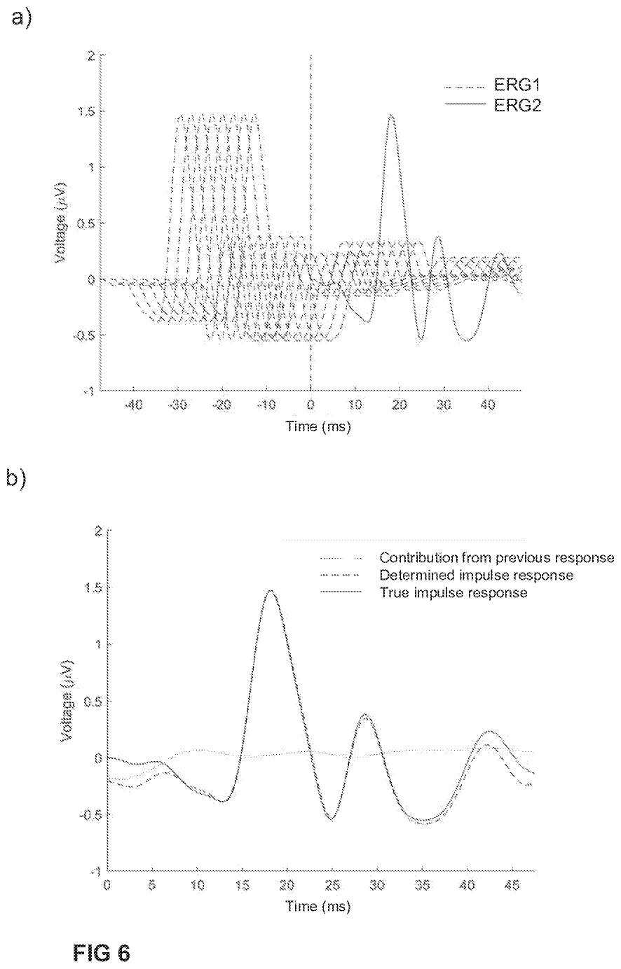

To further demonstrate how the inventive modulation of the stimulus beam affects the determined ERG response, FIG. 5 shows ERG responses obtained with prior art stimulus pulses, while FIG. 6 shows ERG responses obtained with stimulus pulses according to an embodiment of the invention. FIG. 5 shows at a) two separate true or reference ERG responses (electrical responses) that may be elicited using a stimulus beam, a first true ERG response ERG1 and a second true ERG response ERG2. The separate ERG responses are overlaid on the same figure to illustrate how the tail of the previous response (ERG1) overlaps with the subsequent response (ERG2) in the case where stimulating light pulses are provided in an unmodulated manner with even intervals between consecutive light pulses at high frequency.

The consecutive true ERG responses ERG1 and ERG2 overlap in essentially the same phase, resulting in the waveforms of an obtained/recorded ERG signal to be distorted. The ERG signal obtained e.g. around time 0 to 35 ms therefore comprises a sum of the true response to a second stimulus pulse, ERG2, and the tail of the true response to a previous first stimulus pulse, ERG1. This ERG signal will thus be distorted due to the contribution from the previous ERG response.

Therefore, when this distorted ERG signal is used to obtain a determined ERG response, its waveform will also be distorted due to the overlap and the determined ERG response will not correspond to the true ERG response. FIG. 5 shows at b) the true ERG (impulse) response, the determined ERG impulse response, and the contribution in the ERG signal to a following true ERG response ERG2 from the from the previous true ERG response ERG1. It is seen that both the a- and b-waves of the determined ERG response are affected by the contribution from the previous response, such that the determined ERG response differs from the true ERG response.

FIG. 6 depicts at a) a plurality of separate true ERG responses that may be elicited using a stimulus beam, where the separate true responses are overlaid on the same figure to illustrate how the tail of the previous responses (ERG1 now referring to the plurality of ERG responses elicited before the response ERG2) overlap with the subsequent response (ERG2) in the case where stimulating light pulses are provided in a modulated manner with uneven intervals between consecutive light pulses at high frequency.

Here, the consecutive true ERG responses overlap substantially in different phases, whereby the waveform of a recorded ERG signal may be less distorted due to contribution of a tail of a previous response affecting the subsequent response than in the case of unmodulated light pulses delivered at constant frequency.

FIG. 6 b) shows again the true or reference ERG response, the determined ERG impulse response based on an ERG signal that may be recorded utilizing the inventive modulated stimulation, and the contribution in the ERG signal to a following true ERG signal from the previous true response. Due to the recorded ERG signal comprising consecutive true ERG responses that essentially overlap in different phases, the contribution from the previous response is reduced as compared to the case of FIG. 5. The recorded ERG signal and thus the determined ERG response may exhibit less distortion in waveform as compared to the reference or true ERG response and may therefore more accurately represent a true ERG response.

It may be advantageous to obtain a determined ERG response that is closer to an actual ERG response, as any further processing of this determined ERG response may then also be more accurately representative of the actual situation of the retina.

In one embodiment, the ERG signals may be obtained during heating of the target area of the retina with at least two heating powers (of which one may be zero heating power). The determined ERG responses may be used to determine at least one temperature-related parameter corresponding to the at least two heating powers, where the temperature-related parameter is indicative of a temperature of the target area of the retina. The determined temperature-related parameters may be utilized to determine a change in temperature of the target area of the retina that occurs between the two different heating powers.

The kinetics of an ERG response is known to accelerate with rising temperatures. When impulse ERG responses are obtained at a first temperature of the retina and a second temperature of the retina (these temperatures being induced by a first and second heating power), each ERG response signal waveform shifts towards the time of the stimulus pulse an amount that is directly proportional to the time separation between the stimulus and the waveform in question, and the amount of shift is directly proportional to the logarithm of the temperature increase between the first and second temperatures. The temperature difference can be extracted from two ERG responses f₁(t) and f₂(t) by equation 2, where a is the temperature dependence of ERG kinetics acceleration.

$$Δ T = {T}_{1} - {T}_{2} = \frac{1}{α} ln ( {arg max}_{β} ( corr ( {f}_{1} ( \frac{t}{β} ) , {f}_{2} ( t ) ) ) ) \qquad ( 2 )$$

An increase in temperature of the retinal tissue is essentially directly dependent on a power used for the retinal heating. When an increase in temperature is induced with a laser, the temperature increase is then essentially directly dependent on the laser power. Hence, when temperature differences are determined between two determined ERG responses with and without laser exposure, the temperature elevation estimated from the determined ERG responses should be directly proportional to laser power.

However, it was noted by the inventors that upon using prior art stimulus beams with even stimulation intervals between light pulses and sufficiently high stimulation frequency to obtain ERG signals during exposure to two different heating laser powers, and determining ERG responses therefrom to subsequently determine a temperature increase of the retinal tissue, the linear relationship between laser power and temperature of the retinal tissue (through temperature difference determination) did not hold, which will be demonstrated below. A key reason for the inaccuracy in temperature determination with sufficiently high frequency flash stimulation with even flash intervals is that the ERG signaling kinetics caused by temperature elevation changes the phase of the overlapped waveforms. Therefore, the a- and b-waves are affected by both the kinetics acceleration of the of the a- and b-waves themselves, as well as the phase shift of the overlapped waveforms due to temperature elevation.

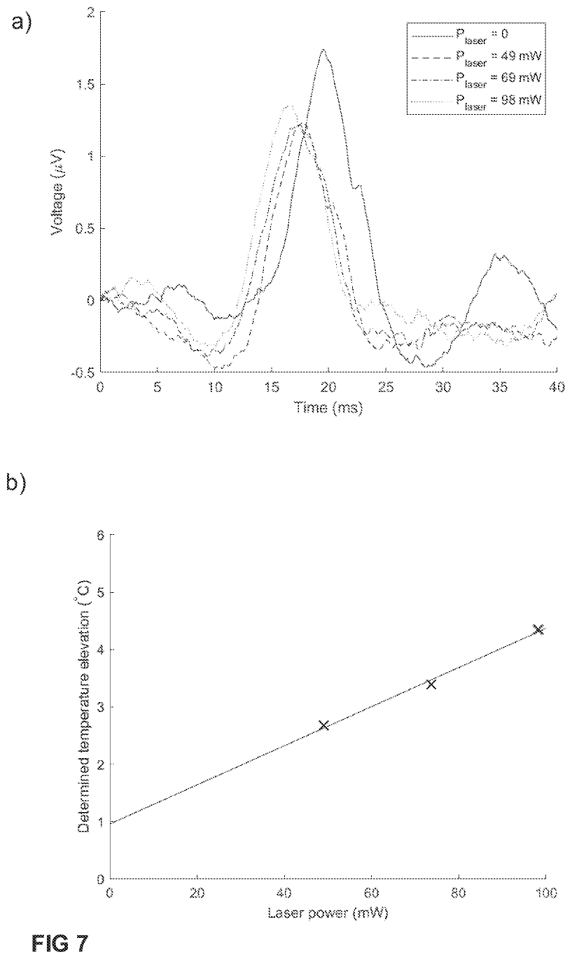

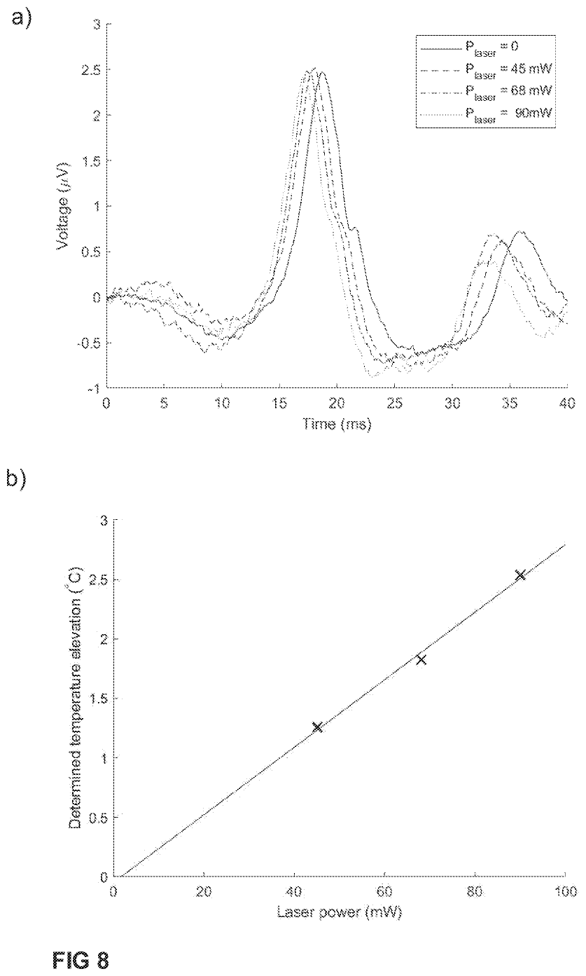

FIG. 7 shows at a) determined ERG responses obtained utilizing ERG signals recorded with 4 laser powers (0 mW, 49 mW, 69 mW, an 98 mW, 3.3 mm laser spot) using unmodulated prior art stimulus light and at b) a determined temperature elevation occurring in the retinal tissue between the different heating laser powers (comparing the laser power of 0 mW to the other used laser powers) based on the determined ERG responses using equation (2). The temperature estimates show nonlinearity with laser power, such that the increase in temperature with increasing laser power appears to change with the temperature, and a linear fit between the different data points does not intersect near the origin. For instance, a two-fold increase in used laser power should double also the temperature increase, while this is not shown in the results of FIG. 7 b), implying error in the temperature determination.

FIG. 8 shows at a) determined ERG responses obtained utilizing ERG signals recorded with 4 laser powers (0 mW, 45 mW, 68 mW, an 90 mW, 5 mm laser spot) with modulated stimulus light pulses according to the invention (with uneven intervals between light pulses) and at b) a determined temperature elevation occurring in the retinal tissue between the different heating laser powers (comparing the laser power of 0 mW to the other used laser powers) based on the determined ERG responses using equation (2). Here, the temperature increase does appear to behave as expected, increasing linearly with laser power, and the linear fit between the data points intersects near the origin. Therefore, the stimulus beam providing modulated light pulses according to the present invention may lead to more accurate determined ERG responses and in particular, more accurate temperature-related parameter determination.

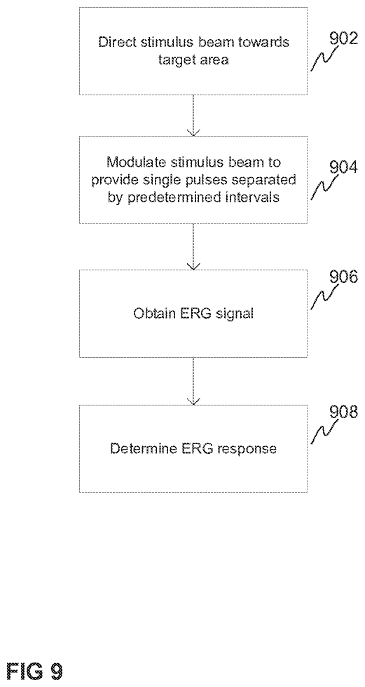

FIG. 9 illustrates a flow chart of a method according to one embodiment of the invention. At 902, a stimulus light beam is directed towards a target area of the retina. The stimulus beam is then modulated 904 to provide a plurality of single pulses of light, wherein consecutive single pulses are separated by predetermined intervals, of which at least a portion differ from each other and/or wherein at least a portion of the single pulses of light comprise predetermined durations of which at least a portion differ from each other.

At least one ERG signal related to an electrical response of the retina is obtained 906, based on which an ERG response of the retina comprising a response of the retinal tissue essentially corresponding to the response of the tissue to at least one of the single pulses based on the ERG signal may be determined 908.

In a further embodiment, the method may additionally comprise directing a heating beam to at least the target area and obtaining at least two measured signals related to an ERG signal of the retina and determining at least one parameter indicative of a change in temperature of the retinal tissue based on the determined ERG responses.

A temperature increase of the retina per unit of heating power may be obtained, and a heating power used for retinal heating based on the determined temperature increase may optionally be controlled.

In another embodiment, the method may additionally comprise removing or reducing artifacts from the obtained ERG signal based on one or more artifact-rejection criteria, where the artifacts relate, for example, to strong, short-lasting muscular activity or burst(s) of external electromagnetic artifacts. The artifacts may be removed from the obtained ERG signal before performing any averaging methods, such as cross-correlation between the ERG signal and a stimulus series used to modulate the stimulus beam.

In a further embodiment, when the intervals and/or durations of the single pulses have been selected such that minimized or reduced signal contribution is provided at or near a mains frequency, such as 50 or 60 Hz, and/or its harmonic frequencies in the frequency band where the ERG signal has spectral content, the method may additionally comprise removing from the analysis (i.e., the determining of an ERG response), or weighting differently, also ERG signal data that would otherwise be considered suitable for the analysis in order to minimize or sufficiently reduce the contribution of noise in mains-related frequencies. The invention has been explained above with reference to the aforementioned embodiments, and several advantages of the invention have been demonstrated. It is clear that the invention is not only restricted to these embodiments, but comprises all possible embodiments within the spirit and scope of inventive thought and the following patent claims.

The features recited in dependent claims are mutually freely combinable unless otherwise explicitly stated.

1. A device for obtaining retinal ERG signals from a target area of the retina, the device comprising means for obtaining an electrical response signal from the target area, the device additionally comprising at least one light source configured to provide a stimulus beam towards the target area, wherein the stimulus beam is modulated to provide a plurality of single pulses of light, where consecutive single pulses are separated by predetermined intervals of which at least a portion differ from each other and/or wherein at least a portion of the single pulses of light comprise predetermined durations of which at least a portion differ from each other.

2. The device of claim 1, wherein the device additionally comprises a heating system, optionally a heating laser, that is configured to elevate the temperature of the target area.

3. The device of claim 1, wherein the intervals and/or durations are selected to provide an ERG signal exhibiting selected overlap of electrical responses that are related to consecutive single pulses.

4. The device of claim 3, wherein the selected overlap comprises overlap of consecutive electrical responses in essentially different phases.

5. The device of claim 3, wherein the selected overlap provides a selected waveform for a determined ERG response obtained from an ERG signal comprising a plurality of overlapped electrical responses through an averaging method, such as cross-correlation between the ERG signal and a stimulus series used to modulate the stimulus beam.

6. The device of claim 5, wherein the selected waveform essentially corresponds to the electrical responses or differs from them by under a threshold amount or wherein the selected waveform essentially corresponds to an obtained reference ERG response or differs from said reference ERG by under a threshold amount.

7. The device of claim 1, wherein the intervals and/or durations are between 10 and 200 ms, advantageously between 20 and 70 milliseconds.

8. The device of claim 1, wherein the intervals and/or durations are selected such that minimized or reduced signal contribution is provided at or near a mains frequency, such as 50 or 60 Hz, and/or its harmonic frequencies in the frequency band where the ERG signal has spectral content.

9. The device of claim 1, wherein the intervals and/or durations and/or an order of the single pulses and associated intervals and/or durations are selected to provide a stimulation pulse train with broad spectral content.

10. The device of claim 1, wherein the longest interval and/or duration is 10 to 150%, advantageously 40 to 100%, longer than the shortest interval and/or duration.

11. The device of claim 1, wherein the intervals and/or durations are approximately evenly spaced between the shortest and the longest interval and/or duration.

12. The device of claim 1, wherein a first set of single pulses of light is provided and an ordering of the single pulses and respective intervals and/or durations is varied at least once to provide at least a second set of single pulses, further wherein said at least second set of single pulses is provided following said first set of single pulses to provide at least one extended set of single pulses of light.

13. The device of claim 1, wherein the single pulses are impulse-like pulses of light or wherein the single pulses are non-impulse-like pulses of light.

14. An arrangement for determining an ERG response, the arrangement comprising the device of claim 1 and at least one processor, the processor being configured to determine a denoised ERG response from an obtained ERG signal comprising overlapping ERG responses.

15. The arrangement of claim 14, wherein the device additionally comprises a heating system, optionally a heating laser, that is configured to elevate the temperature of the target area, wherein the processor is further configured to additionally determine at least one parameter indicative of a change in temperature of the retinal tissue based on determined ERG responses.

16. A method for determining an ERG response, the method comprising
directing a stimulus light beam towards a target area of the retina
modulating the stimulus beam to provide a plurality of single pulses of light, wherein consecutive single pulses are separated by predetermined intervals, of which at least a portion differ from each other and/or wherein at least a portion of the single pulses of light comprise predetermined durations of which at least a portion differ from each other,
obtaining at least one ERG signal related to an electrical response of the retina,
determining an ERG response of the retina comprising a response of the retinal tissue essentially corresponding to the response of the tissue to at least one of the single pulses based on the ERG signal.

17. The method of claim 16, wherein the method additionally comprises directing a heating beam to at least the target area and obtaining at least two measured signals related to an ERG signal of the retina and determining at least one parameter indicative of a change in temperature of the retinal tissue based on the determined ERG responses.

18. The method of claim 16, wherein the method comprises determining a temperature elevation of the retina per unit of heating power and optionally controlling a heating power used for retinal heating based on the determined temperature elevation of the retina per unit of heating power.

19. The method of claim 16, wherein the method additionally comprises removing or reducing artifacts from the obtained ERG signal based on one or more artifact-rejection criteria.

20. The method of claim 19, wherein, when the intervals and/or durations of the single pulses have been selected such that minimized or reduced signal contribution is provided at or near a mains frequency, such as 50 or 60 Hz, and/or its harmonic frequencies in the frequency band where the ERG signal has spectral content, the method additionally comprises removing from the determining of an ERG response or weighting differently also ERG signal data that would otherwise be considered suitable for the analysis such that the contribution of noise in mains-related frequencies is minimized or sufficiently reduced.
